(19) United States
(12) Patent Application Publication
(10) Pub. No.: US 2013/0042615 A1
(43) Pub. Date: Feb. 21, 2013

(54) Advanced method and apparatus for addressing the serious pollution from existing coal-burning power stations

(71) Applicants: Albert Calderon, Bowling Green, OH (US); Richard Owen McCarthy, Strongsville, OH (US); Terry James Laubis, Portage, OH (US)

(72) Inventors: Albert Calderon, Bowling Green, OH (US); Richard Owen McCarthy, Strongsville, OH (US); Terry James Laubis, Portage, OH (US)

(21) Appl. No.: 13/199,034

(22) Filed: Aug. 18, 2011

(51) Int. Cl. F01K 13/00; B01D 53/74; B01D 53/50; B01D 53/56; C22C 7/00; C01B 3/02; B01J 19/00; B01J 7/00; C07C 5/00; C03C 3/00; B01D 53/62; C07C 41/01

(52) U.S. Cl. 60/648; 423/230; 423/210; 423/242.1; 423/239.1; 568/671; 423/648.1; 422/187; 48/61; 585/700; 501/11; 75/392

(57) ABSTRACT

The present invention deals with the serious pollution problems from electric power plants that burn coal which may be forced to shut down by virtue of their being uneconomical to be retrofitted with expensive pollution controls. The pollutants from coal-burning power stations comprise SO₂, NOₓ, Hg, Particulate Matter, Ash, and CO₂. This invention offers a unique and comprehensive solution that makes possible the prevention of the ill-effects currently caused to health and environment while at the same time would also prevent the closure of these badly needed power generation facilities that provide some 50% of the electricity generated in this country. The herein comprehensive solution converts the six mentioned pollutants into valuable products and thus avoids the discharge of such pollutants into the atmosphere.

The present invention is targeted to solving the serious pollution problems originating from the generation of electric power from plants which burn coal that may be forced to shut down by virtue of their being uneconomical to be retrofitted with expensive pollution controls; see Exhibit 1. The pollutants from coal-burning power stations comprise SO₂, NOₓ, Hg, Particulate Matter, Ash, and CO₂. This invention offers a unique and comprehensive solution which makes possible the prevention of the ill-effects currently caused to health and environment while at the same time would also prevent the closure of these badly needed power generation facilities that provide some 50% of the electricity generated in this country whose citizens so heavily depend on. In addition to the herein comprehensive solution, it will be disclosed in the specification that follows, the putting of all six pollutants mentioned above into beneficial use while avoiding the discharge of said pollutants into the atmosphere.

### BACKGROUND

The renowned Clean Air Task Force (CATF), whose main office is in Boston, Mass., with several branches, issued in September 2010 a Report titled “The Toll From Coal” and subtitled “An Updated Assessment of Death and Disease from America's Dirtiest Energy Source.” The first paragraph of the Report's Executive Summary states the following:
“Among all industrial sources of air pollution, none poses greater risks to human health and the environment than coal-fired power plants. Emissions from coal-fired power plants contribute to global warming, ozone smog, acid rain, regional haze, and—perhaps most consequential of all from a public health standpoint—fine particle pollution. In 2000 and again in 2004, the Clean Air Task Force commissioned comprehensive studies of health impacts caused by fine particle air pollution from the nation's roughly 500 coal-fired power plants. Each study incorporated the latest scientific findings concerning the link between air pollution and public health, as well as up-to-date emissions information. Both found that emissions from the U.S. power sector cause tens of thousands of premature deaths each year and hundreds of thousands of heart attacks, asthma attacks, emergency room visits, hospital admissions, and lost workdays.”
   Further, on page 8 of the Report, the first six lines of the 3^{rd} paragraph state the following:
“Unfortunately, persistently elevated levels of fine particle pollution are common across wide swaths of the country, particularly in the eastern United States. Fine particle pollution itself consists of a complex mixture of harmful pollutants including elements as diverse as soot, acid droplets, and metals. Most of these pollutants originate from combustion sources such as power plants, diesel trucks, buses, and cars.”

### OBJECTIVES

The main object of the present invention is to avoid the burning of coal in boilers of existing electric power stations by efficiently processing the coal upstream of the boilers in an environmentally closed system while producing clean gases that are utilized to generate clean, low-cost power as well as valuable by-products.

Another object of the instant invention is to prevent layoffs from shutting down coal-burning power generating facilities and, instead, create many additional well-paying jobs.

Therefore another object of the present invention is to capitalize on existing infrastructure in the power stations that is quite costly to replace.

Yet another object of the instant invention is to create energy security by providing ample capacity to prevent black-outs.

Further another object of the present invention is to provide one comprehensive solution that will control SO₂, NOₓ, Hg, Particulate Matter, Ash, and CO₂, from coal.

Still another object of the instant invention is to eliminate the need for pulverizing the coal, as pulverization is notorious in producing fine particulate matter that is injurious to health.

Further still another object of the present invention is to increase the availability of boilers currently used in coal-burning power stations, by avoiding the burning of coal altogether in boilers, which currently demand frequent maintenance caused by deposits within the boilers as a result of combusting coal in the boilers.

Further yet another object of the present invention is to generate clean electric power more efficiently while still using existing boilers to raise steam that can serve as the steam cycle of a hybrid, efficient combined cycle power generation.

It is therefore another object of the instant invention to increase capacity of power generation with low capital investment.

It is yet another object of the instant invention to increase the net profit of power producers using clean gases made from coal, which will enable such producers to offer attractive power costs to the consumer.

Other objects of this invention will appear from the following detailed description and appended claims. Reference is made to the accompanying drawings forming a part of this specification wherein like reference characters designate corresponding parts in the various figures.

### BRIEF DESCRIPTION OF THE DRAWINGS

FIG. 1 is a general layout of the present invention.

FIG. 2 illustrates a pyrolyzing reactor in perspective which can efficiently process the coal in the form of bituminous, sub-bituminous, lignite, or peat, including a frontal view of a cross-section along the longitudinal axis of the pyrolyzing reactor shown in FIG. 2.

FIG. 3 is an enlarged, partial, longitudinal section of the pyrolyzing reactor, including a cross-section view taken at A-A of FIG. 3.

FIG. 4 illustrates the trapping of mercury from coal-derived gases by means of activated carbon (char) but with the addition of the recovery of elemental mercury (Hg) from the mercurized activated carbon.

FIG. 5 illustrates the end view of several pyrolyzing reactors assembled together in battery form to satisfy large production needs.

### DETAILED DESCRIPTION OF THE DRAWINGS

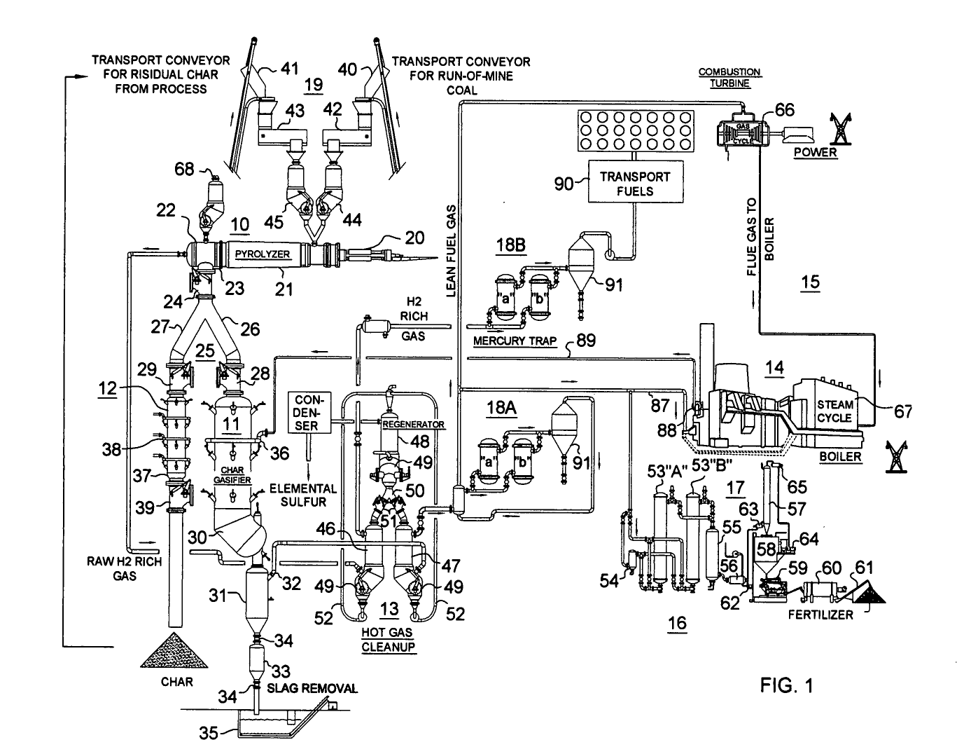

Reference is made to FIG. 1 wherein the following numerals represent the main components: 10 marks the pyrolyzer; 11 marks the char gasifier; 12 marks the char quencher; 13, the hot gas cleanup; 14, the existing coal-burning boiler; 15, the combined cycle electric power generation; 16, the alternating reducing reactors that produce the intermediate feedstock comprising the upstream portion of the fertilizer plant; 17, the fertilizer (oxamide) reactor; 18A and 18B, the dual beds of activated carbon for mercury removal; and 19 is the equipment to feed the run-of-mine coal and residual char as fuel into pyrolyzer 10.

Pyrolyzer 10 is made up of charger 20, pyrolyzing chamber 21, radiant zone 22, downcomer 23, and flow control valve 24, from which bifurcated pipe 25 forms a delivery pipe assembly, with pipe 26 connecting downcomer 23 thence to char gasifier 11 by way of control valve 28, and pipe 27 connecting downcomer 23 to char quencher 12 by way of control valve 29. It is to be noted that gasifier 11 serves to perform three functions; namely, the conversion of hot incandescent residual char into a raw lean gas, the reduction of CO₂ into 2CO (a fuel or chemical feedstock), and the conversion of coal ash (a polluter) into inert slag.

Gasifier 11 comprises vessel 30, which is equipped with injection points at different levels for a gas containing oxygen, such as air, to react with hot char to produce a lean fuel gas; gasifier 11 possesses at its bottom discharge cooler 31 that is equipped with a lean gas exit port 32; cooler 31 serves to solidify molten slag produced in gasifier 11 into an inert grit. Below cooler 31, lockhopper 33 is provided, which is controlled by upper valve 34 and by lower valve 34 that controls the discharge of the solidified, inert slag into collection tank 35. At about mid-point of gasifier 11, a special manifold marked by numeral 36 serves for the injection of flue gases containing CO₂ for reducing the CO₂ into 2CO that serves as a feedstock. Char quencher 12 comprises vessel 37, which is equipped with multi-level manifolds, like manifold 38, that gradually cool the char below ignition point prior to being periodically discharged to the atmosphere by means of valve 39.

The equipment to feed the run-of-mine coal and the char, marked by numeral 19, comprises skip 40 which elevates the run-of-mine coal from ground level to conveyor 42 and skip 41 which elevates the char (fuel) from ground level to conveyor 43, which in turn conveyor 42 discharges to feeder 44 and conveyor 43 discharges to feeder 45. A secondary surge hopper marked by numeral 68 serves to feed polluted boiler ash from coal combustion that had been stored in ponds (Exhibit 2) and classified as hazardous material. In feeding such hazardous material into gasifier 11, such ash is caused to mix with the ash from the freshly fed char from pyrolyzer 10 into slagging gasifier 11, thus providing a way of converting the old, hazardous ash and the newly formed ash into inert slag.

Gas cleanup 13 is made up of three vessels, marked by numerals 46, 47, and 48. Vessel 46 cracks and simultaneously desulfurizes the H₂ rich gas (volatile matter) from pyrolyzer 10; vessel 47 cleans the lean fuel gas made up of nitrogen (N₂) and carbon monoxide (CO) gas from gasifier 11; and vessel 48 serves to regenerate the spent sorbent while producing elemental sulfur directly and additional lean fuel gas. All three vessels are equipped with feeders denoted by numeral 49. Vessel 48 interconnects with vessels 46 and 47 via the inverted Y-pipe that is marked by numeral 50, which is equipped with diversion valves 51. Gas cleanup 13 is equipped with pneumatic transporters 52 to convey the spent sorbent from vessels 46 and 47 to regenerator 48.

Cyanogen make-up equipment 16 comprises reactor 53 “A” and reactor 53 “B” with gas temperature moderator denoted by numeral 54 being upstream of reactors “A” and “B,” and chiller-liquefier which is denoted by numeral 55, being downstream of reactors 53A and 53B. A separator marked by numeral 56 is provided to segregate the liquefied cyanogen from the unreacted gases which are directed (not shown) to pyrolyzer 10, or recycled back to either reactor 53A or 53B.

Downstream of separator 56, oxamide maker 17 is located. It consists of reactor 57, settling tank 58, filter press 59, drier 60, and stacker 61. Pump 62 is provided to separator 56, to pump the liquefied cyanogen to evaporator 63, and pump 64 serves to circulate the liquid catalyst to the top of reactor 57; a heater denoted by numeral 65 serves to adjust the temperature of the liquid catalyst.

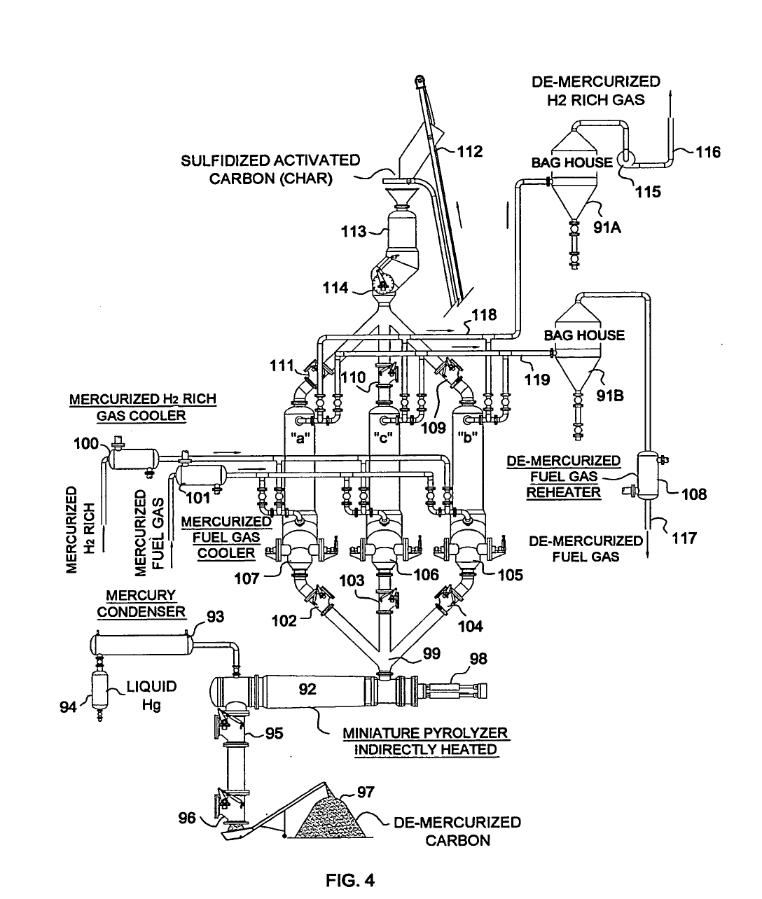

The mercury removal systems marked by numeral 18A and 18B (also known as mercury traps) consist of activated carbon beds, comprising beds “a” and “b,” with the practice being when bed “a” is in absorption of mercury, bed “b” is in stand-by mode, and when bed “b” is in absorption, while bed “a” is in stand-by mode. A baghouse marked by numeral 91 is provided downstream of each mercury trap. A preferred configuration of mercury capture is described hereinafter in FIG. 4, wherein the recovery of elemental mercury from saturated activated carbon is effected.

The electric power generation system in this invention, marked by numeral 15, is preferably fueled with a clean, lean gas (fuel gas) fed from cleanup vessel 47 and comprises combustion turbine numeral 66 connected to existing boiler 67 which serves as both—a heat recovery steam generator and steam turbine by advantageously making use of the very valuable coal-burning boiler 67, by jointly forming a combined cycle configuration by making use of combustion turbine 66; such configuration provides a most efficient way of generating power while still salvaging the existing coal-burning boiler but without combusting coal in it, to raise steam by means of hot exhaust from combustion turbine 66. If the supply of the hot exhaust is inadequate, clean lean gas (2CO+N₂) is fed to the boiler by means of duct 87 as clean supplemental fuel. Instead of discharging the flue gas (N2+CO₂) from boiler 67 into the atmosphere, the flue gas is compressed by means of compressor 88 and fed to gasifier 11 for reduction of the CO₂ to 2CO₃ by means of duct 89, which ties to manifold 36 of gasifier 11, thus providing a completely closed-to-the-atmosphere system.

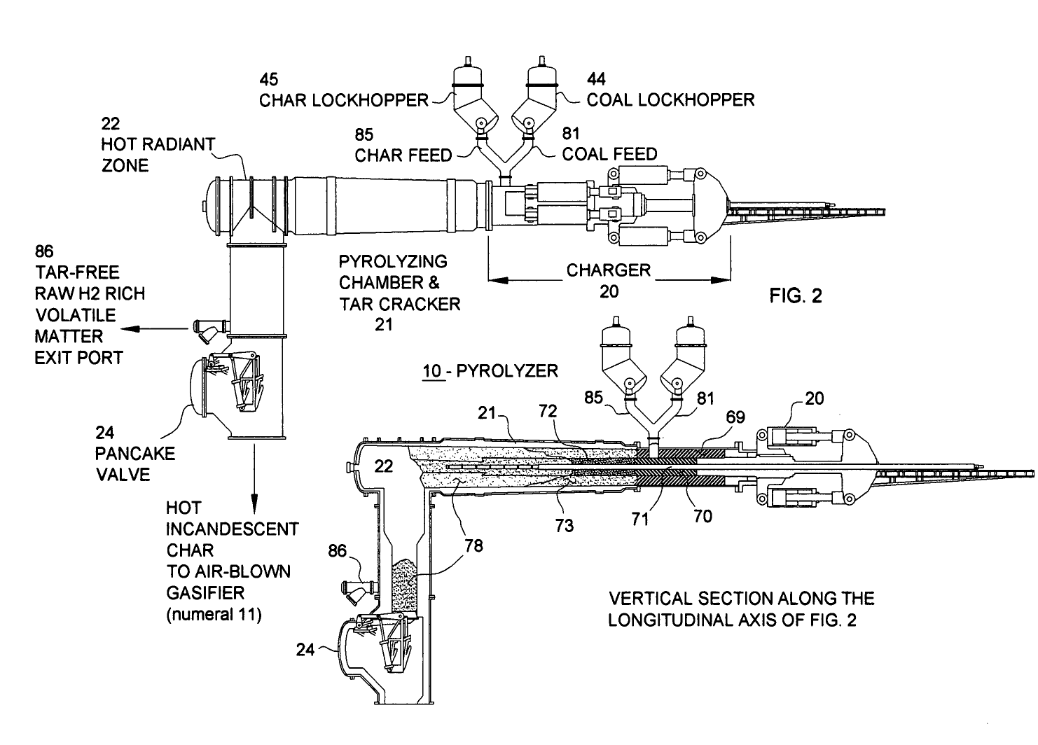

FIG. 2 illustrates in perspective the pyrolyzer denoted by numeral 10 and is made up of feeders 44 and 45, charger 20, pyrolyzing chamber denoted by numeral 21, radiant zone 22, and control valve 24. The coal and the char are fed by way of pipes 81 and 85, respectively, using a “Y” piping configuration. The exit port for the H₂ rich volatile matter is marked by numeral 86.

For additional clarification, pyrolyzer 10 is illustrated in a vertical, frontal section taken along the longitudinal axis of FIG. 2, to show the internals of pyrolyzer 10. It is to be noted that in providing lance 71 wherein the char charged as a core denoted by numeral 72 is combusted under suppressed conditions (in a pressurized, controlled reducing atmosphere), the heat transfer within chamber 21 is markedly improved, thus enhancing the rate at which the coal devolatilizes into volatile matter, while vigorously cracking unwanted tars.

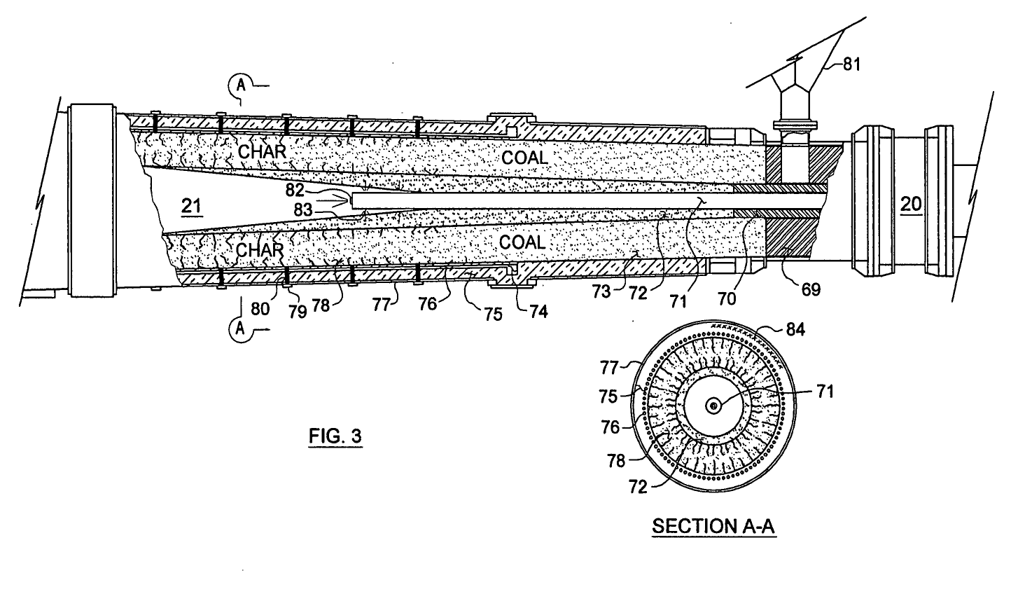

Referring now to FIG. 3 for additional detail at a larger scale of pyrolyzer 10, lance 71, in addition to its capability to inject oxygen through its tip denoted by numeral 82, is equipped with injection nozzles on its side denoted by numeral 83. Lance 71, like mandrel 70 and ram 69, is adapted to advance and retract independently, and because of the high temperature surrounding lance 71, it is cooled preferably with water circulating through it in a closed loop.

The coal is heated peripherally by means of injection nozzles disposed through shell 77 and refractory 75, one of which being marked by numeral 80, with such nozzles being supplied with a gas containing oxygen furnished by manifold 79, thus providing direct, pressurized, bi-directional, efficient heating that increases the release of the volatile matter from the coal annulus to such an extent that virtually all the oils in the coal are recovered in tar-free vapor form.

In the instant invention, wherein a core of char is surrounded by an annulus of coal and the char is combusted, thus minimizing the combustion of coal, the yield of H₂ rich raw gas (raw syngas) is markedly increased, this being an important and beneficial factor, as a H₂ rich syngas is quite valuable to be used to produce chemicals and transportation fuels such as methanol/gasoline and dimethyl ether, a clean fuel, as a substitute for dirty diesel. To achieve this objective, numeral 21 is the pyrolyzing chamber, numeral 20 is the charger, numeral 81 is the feed hopper, numeral 69 is the ram, numeral 70 is the mandrel, numeral 71 is the injection lance, numeral 82 is the nozzle at the tip of lance 71, and numeral 83 is one of the several nozzles disposed at the side of lance 71, numeral 72 is the char fuel, numeral 73 is the charged coal, and numeral 75 is the refractory/insulation which is configured as a monolithic structure that is reinforced with metallic needles such as stainless steel needles, marked by numeral 84 (shown in SECTION A-A), somewhat similar to imbedding steel wire in reinforced concrete; this structure is cast in place against shell 77.

In the case of heating the material peripherally directly by combusting char (not shown), oxygen is introduced through shell 77 by means of injectors, one such injector being marked by numeral 80 supplied by manifold 79. When combustion takes place peripherally around the coal annulus, it is possible to also charge char around the perimeter of the coal annulus by providing an additional mandrel that circumscribes ram 69 to form a ring of char around the periphery of the coal. In so doing, the combustion effected by injectors, such as injector 80, consumes the ring of char, instead of combusting the coal, as the objective is to maximize the production of H₂ rich gas, which after cleanup, is a very valuable feedstock.

In the case of heating the material peripherally indirectly, numeral 74 represents the manifold for distributing hot heating gas into a plurality of small-diameter flues installed in refractory/insulation 75, one such flue being marked by numeral 76 carrying hot gases that heat refractory 75, which in turn heats indirectly the coal marked my numeral 78 shown in Section A-A. It is to be noted that towards the exit end of pyrolyzing chamber 21, the coal has been completely devolatilized, yielding a residue consisting of hot incandescent char.

Referring to FIG. 4, which illustrates the preferred way of trapping mercury and recovering the mercury as elemental mercury, a very valuable product, the Figure consists of activated carbon beds “a,” “b,” and “c.” By way of example, bed “a” would de-mercurize the H₂ rich gas, and bed “b” would de-mercurize the fuel gas; prior to the gases being de-mercurized, the H₂ rich gas is passed through cooler 100, and the fuel gases being de-mercurized, the H₂ rich gas is passed through cooler 100, and the fuel gas is passed through cooler 101 to drop the temperature of the gases to render the de-mercurization of the gases effective, as de-mercurization takes place at low temperature, with the H₂ rich gas fed to bed “a” and the fuel gas to bed “b”; bed “c” is provided in the configuration to serve as an alternate bed for back-up when bed “a” or bed “b” requires maintenance.

Beds “a” and “b” get charged from the top with activated carbon, with flow control valves 111 and 109, respectively; beds “a’ and “b” get discharged from the bottom, with flow control valves 102 and 104, respectively. Upstream of valves 102 and 104, feeders 107 and 105 are situated in such a way that feeder 107 is upstream of valve 102, and feeder 105 is upstream of value 104. Upstream of flow control valves 111 and 109, common feeder 114 is disposed to enable the feed of fresh activated carbon to either bed “a” or bed “b,” with common feeder 114 forming the lower portion of surge hopper 113, which serves as storage for fresh activated carbon; surge hopper 113 receives activated carbon by means of skip hoist 112, which elevates the activated carbon from ground level.

To regenerate the saturated (mercurized) carbon from beds “a” and “b,” valves 102 and 104 discharge the mercurized carbon into the charging chamber of miniature pyrolyzer 92 by way of manifold 99, within which the mercurized carbon is heated indirectly, causing the vaporization of the mercury which is directed from pyrolyzer 92 to condenser 93 where the recovered mercury is cooled and collected in liquid form in tank 94. The feed of the mercurized carbon through pyrolyzer 92 is effected by a ram pusher marked by numeral 98, and the de-mercurized carbon is discharged from pyrolyzer 92 by means of lockhopper having an upper valve marked by numeral 95 and a lower valve marked by numeral 96, while the de-mercurized carbon is noted by numeral 97.

Downstream of beds “a” and “b,” two baghouses are disposed and respectively marked by numerals 91A and 91B, with baghouse 91A serving to clean particulate matter entrained in de-mercurized H₂ rich gas stream 118, and baghouse 91 B serving to clean particulate matter entrained in de-mercurized fuel gas stream 119. Subsequent to the removal of particulate matter from stream 118, the cleaned, de-mercurized H₂ rich gas is raised in pressure by means of compressor 115, forming stream 116 which is directed to plant 90 (shown in FIG. 1) to produce a chemical such as methanol that can be converted to gasoline or dimethyl ether; subsequent to the removal of particulate matter from stream 119, the cleaned, de-mercurized fuel gas is raised in temperature in heater 108, forming stream 117 which is directed to combustion turbine 66 (shown in FIG. 1) to generate electric power. As stated above, activated carbon bed “c” can take the place of activated carbon bed “a” or activated carbon bed “b,” as its purpose serves to substitute bed “a” or bed “b” when either one of these beds is down for maintenance.

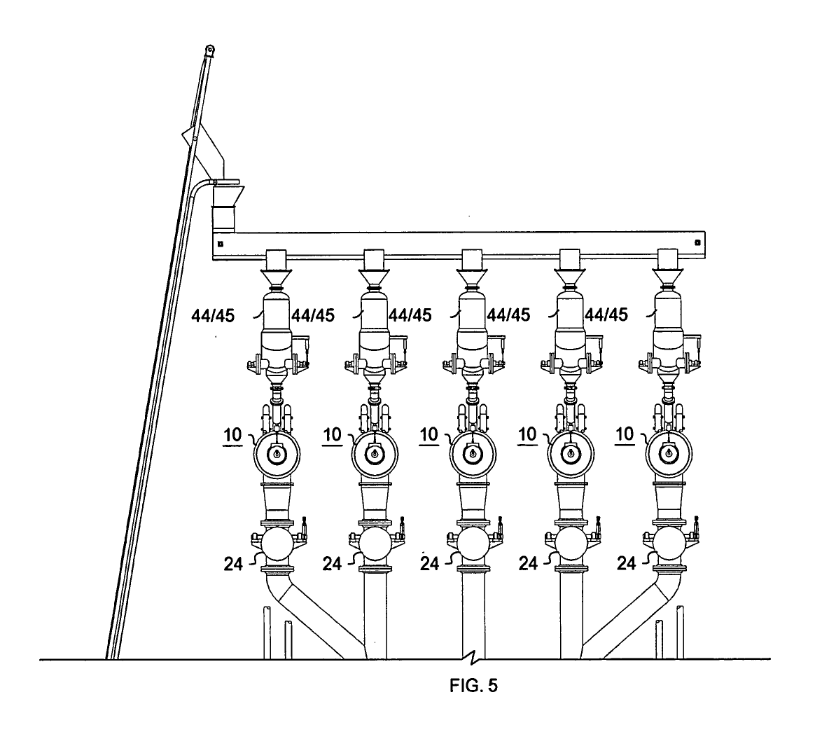

Referring to FIG. 5, it illustrates a group of pyrolyzers configured in battery form to provide a modular structure in order to enable it to efficiently scale-up productive capacity by replication.

### Operation

To describe the operation of this invention based on extensive test work that had taken place and referenced hereinafter begins with using unprepared, crushed run-of-mine coal preferably of three inches and under that is directly fed into a battery of pyrolyzers, where the cracking of asphalt in highly hot radiant zone 22 results in a tar-free volatile matter containing a hydrogen rich, non-condensable raw syngas together with vaporized light liquids and incandescent char. The syngas and vaporized light liquids are desulfurized and upgraded in the first hot gas cleanup 46 (shown in FIG. 1), producing a clean syngas suitable to make chemicals or fuels such as methanol converted to gasoline or to dimethyl ether represented by numeral 90 in FIG. 1. With respect to the hot char, a part of the hot char is gasified with air in gasifier 11, producing a raw fuel gas; the other part of the char is used as fuel for heating the pyrolyzer and two additional good uses; namely, (i) char which is activated with steam and used to trap Hg; and (ii) char used as carbon enrichment of soil mixed with the fertilizer to convert it to a super-fertilizer. The raw fuel gas from gasifier 11 is passed through a second hot gas cleanup 47 (shown in FIG. 1), producing a clean, desulfurized lean gas which is ideal to generate clean, efficient electric power with the emitted N₂+CO₂ from power generation collected and converted to N₂+2CO₃ which serves as a feedstock to make a slow-release fertilizer, a most valuable by-product.

The test work performed in the Applicant's pilot in cooperation with Sun refining proved that the method described herein, which uses CaO as sorbent, produced light liquids from cracking residuum (heavy bitumen) from its Philadelphia Refinery against CaO as sorbent. Such light liquids were referred to by Sun as “excellent feedstocks and can be separated by a simple distillation process into valuable intermediates”; see Exhibit 3, page 1 of 2.

Data that was produced by way of the tests (see Exhibit 3, page 2 of 2) showed that the Ramsbottom Carbon (by weight percentage) of the residuum was converted from 18.2% to 1.24% in test Run #3, to 0.59% in test Run #4, and to 0.31% in test Run #5. Further, for the Pour Point temperature in ° F., the residuum was 145° F., and in tests #3, #4, and #5, the temperature was reduced to −20° F. Also, the INITIAL BOILING POINT of the residuum dropped from 802° F.: in test #3 to 108° F., in test #4 to 154° F., and in test #5 to 135° F. This data show that the method herein described—which is based on the replication of the test work performed, except at commercial scale—should produce outstanding results in producing light liquids from bituminous coal. It is also important to disclose herein that the syngas (Rich Gas Sample—Test Run #3) produced in Mole % as follows: H₂—57.3%; CH₄—36.6%; N₂—3%; C₂H₄—1.8%; CO 1.6%; and CO₂—only 0.7%.

Additional work with respect to residual char after pyrolysis, tests were conducted in 1997 at Applicant's Process Development Unit (Exhibit 4) making metallurgical coke from coal; the coke (char) produced was tested for various properties including residual volatile matter after pyrolysis. In testing the coke made from Bethlehem Steel's coal, the residual volatile matter in the coke was 0.58%, and with coke made from U.S. Steel's three coals, the residual volatile matter in the coke was 0.55% from Blue Tag Coal, 0.48% from Low-Vol coal, and 0.70% from White Tag coal; see Exhibit 5. In the tests conducted, whether the feedstock was heavy oil (bitumen) or coal, these feedstocks were pyrolyzed in sealed tubes in which cracking of tars took place as proposed herein; in the case of sulfur removal, the gas produced had no H₂S, as reported in Exhibit 3, page 1 of 2. Elemental sulfur was produced directly during regeneration (see Exhibit 6), and the chemistry for such results were published in The Making, Shaping and Treating of Steel, 11^{th} edition; see Exhibit 7.

As referenced above, there are six pollutants as a result of combusting coal in existing coal-fired boilers to raise steam which is fed into steam turbines to generate electric power. These pollutants consist of: SO₂, NOₓ, Hg, Particulate Matter, Ash, and CO₂. The comprehensive solution of the instant invention is to convert all the six pollutants into valuable products instead of wastes being discharged into the atmosphere or buried in landfills or some geologic formation which is costly, inefficient, and must be continuously monitored.

The herein invention addresses these six pollutants into products as follows:
1. Sulfur Dioxide (SO₂)—Sulfur is quite common as an inherent component of coal which when combusted becomes SO₂. By not combusting the coal but pyrolyzing it, the sulfur takes the form of H₂S (see Exhibit 3) that reacts with CaO in hot gas cleanup vessel 46 to become carbon-impregnated CaS which, when regenerated, the sulfur is released as elemental sulfur, a valuable by-product.
2. Oxide of N₂ (NOₓ)—When combusting coal in a boiler, NOₓ is formed. By not combusting coal in a boiler and substituting a hot exhaust gas from a combustion turbine which combusts a clean, lean fuel gas (LFG gas [Low Btu Fuel gas]), the amount of NOₓ formed is about 10 parts/million (ppm) which, when compared to the combustion of natural gas (CH₄), is considered very clean by industry and by the general public; its NOₓ production is 152 ppm, 5 which is equal to some 15 times greater than the NOₓ produced from combusting low Btu Fuel Gas; see Exhibit 8. Even though the NOₓ is quite low (10 ppm), the flue gas in the application of the technology disclosed herein is not discharged into the atmosphere, as will be explained hereinafter while addressing the issue of CO₂, since the flue gas also contains CO₂.
3. Mercury capture (Hg)—The mercury trap (Exhibit 9) is adapted to remove the mercury by means of sulfidated activated carbon (char) made in-house wherein mercurized H₂ rich gas is de-mercurized through a bed “a” and mercurized fuel gas is de-mercurized through a bed “b” while a third bed “c” is provided to relieve bed “a” or bed “b” when either bed “a” or bed “b” becomes saturated with Hg, as illustrated in FIG. 4. The de-mercurized H₂ rich gas from bed “a” is fed through baghouse 91A, thence to the methanol plant for conversion to gasoline or dimethyl ether. With respect to the de-mercurized fuel gas, it is fed through baghouse 91B (FIG. 4), thence fed to combustion turbine 66 (FIG. 1) to generate power, with its hot exhaust being directed through boiler 67 to raise steam and generate power. The herein equipment is configured in such a way as to be capable to substitute bed “a” or bed “b” with bed “c” when either bed “a” or bed “b” is saturated with mercury. To recover the mercury as a valuable by-product, a miniature pyrolyzer, which is provided as part of the mercury recovery system, is indirectly heated to vaporize the mercury from the carbon and be separated from the carbon by way of condensation. The separated mercury in vapor form is cooled in a condenser to convert it into valuable, pure mercury in liquid form.
4. Particulate Matter—In the case of particulate matter, the coal is not pulverized, but it is used as delivered from the mine. Within the method herein described, the coal pyrolysis and hot gas cleanup of both the H₂ rich gas and the lean fuel gas are continuously maintained in an environment which is closed to the atmosphere to physically eliminate emissions. The particulate matter is collected in cyclones and baghouses and delivered to gasifier 11 where it is melted, with the ash forming a glassy, inert slag
   In the conversion of the H₂ rich gas into methanol/gasoline or dimethyl ether, the system is completely closed, including the stored product in tankage ready for shipment to customers. In the case of the lean fuel gas, it is completely closed until electric power is delivered to the switch yard whence it is transmitted to the grid. 5. Coal Ash—The method herein described integrates the pyrolysis of the coal in pyrolyzer 10, producing a H₂ rich gas and a hot incandescent char which is efficiently gasified in gasifier 11 which melts the ash in the coal to produce a glassy, inert slag by operating at such a high enough temperature to insure that the constituents of the ash are fully melted; various analyses of the slag produced by the Applicant in cooperation with Sun Oil Company are shown in Exhibit 10.
6. Carbon Dioxide (CO₂)—The carbon dioxide is formed during the combustion of the lean fuel gas with air in gas turbine 66. When lean fuel gas is also combusted in boiler 67 as a supplement for additional thermal energy input, the energy from the hot exhaust flue gas directed from gas turbine 66 to boiler 67 is augmented. The total flue gas leaving boiler 67, whose composition is N₂+CO₂, is fed by means of stream 89 to gasifier 11 where the N₂+CO₂ is converted by the hot char in gasifier 11 to N₂+2CO and becomes part of the lean fuel gas that leaves via port 32 of the hot gas cleanup. Thus, the CO₂, instead of being captured and sequestered in a geologic formation, is converted to a feedstock to produce fertilizer, a very valuable product.

In conclusion, based on the test work done and the herein description, the objectives listed towards the beginning of this disclosure are achievable. It is submitted herein that the instant method and apparatus provide major improvements over the conventional practice of using pulverized coal that is combusted in boilers. The details of construction mentioned above are for the purpose of description and not limitation, since other configurations are possible without departing from the spirit of the invention. Further, other materials besides coal can be processed in the apparatus herein described.

We claim:

1. In a method of generating thermal energy from coal such as by combusting coal in boilers to raise steam which is fed into steam turbines to generate electric power wherein pollutants in the form of SO₂, NOₓ, Hg, particulate matter, ash, and CO₂ are produced, the improvement of providing a comprehensive solution that integrates all the streams that make up said pollutants, in a closed manner which prevents any one of said pollutants to be discharged into the atmosphere, while converting said pollutants into useful, valuable products.

2. The method as set forth in claim 1 wherein said SO₂ is prevented from being discharged into the atmosphere but converted to elemental sulfur, a valuable, marketable product.

3. The method as set forth in claim 1 wherein said NOₓ is prevented from being discharged to the atmosphere and converted by reaction with hot char to become N₂+2CO, a feedstock for producing fertilizer, a valuable and marketable product.

4. The method as set forth in claim 1 wherein said Hg is prevented from being discharged into the atmosphere and recovered in the form of elemental Hg, a valuable, marketable product.

5. The method as set forth in claim 1 wherein said particulate matter is prevented from being discharged into the atmosphere and instead collecting the particulate matter in baghouses and feeding it into a gasifier which has the capability of converting such particulate matter into a glassy, inert slag that can be marketed as a clean aggregate.

6. The method as set forth in claim 1 wherein said ash is prevented from being stored in ponds by converting said ash into a glassy, inert slag that can be marketed as a clean aggregate.

7. The method as set forth in claim 1 wherein said CO₂ which is contained in flue gas (N₂+CO₂) is reduced with hot char to N₂+2CO that serves as a feedstock to make fertilizer, a valuable, marketable product.

8. The method as set forth in claim 1 wherein coal is pyrolyzed to produce a hydrogen rich gas which after cleanup and de-mercurization is converted to transportation fuel to replace petroleum-based fuel.

9. The method as set forth in claim 8 wherein said transportation fuel is gasoline or dimethyl ether.

10. The method as set forth in claim 8 wherein said hydrogen rich gas which after cleanup and de-mercurization is used as a chemical feedstock.

11. The method as set forth in claim 1 wherein coal is pyrolyzed to produce a char which is gasified with a gas containing oxygen which converts said char to a fuel gas.

12. The method as set forth in claim 11 wherein said gas containing oxygen is air to produce a lean fuel gas which when combusted inherently produces low amounts of NOₓ, much lower than when natural gas is combusted.

13. The method as set forth in claim 1 wherein coal is pyrolyzed to produce a char which is fed directly into an integrated char gasifier.

14. The method as set forth in claim 13 wherein polluting ash from ponds is fed into said char gasifier to convert such ash into a glassy, inert slag.

15. The method as set forth in claim 1 includes apparatus to perform the functions described in claim 1.

16. The apparatus referenced in claim 15 wherein said apparatus comprises the following:
means to transport coal and other materials from ground level and to feed such coal and materials;
a pyrolyzation means adapted to devolatilize coal to produce volatile matter and hot char;
means to separate said volatile matter from said char;
hot gas cleanup means to desulfurize and crack hydrocarbons in said volatile matter to produce a hydrogen rich gas (syngas);
char gasifier means to convert said char into a lean fuel gas while converting the ash in the char to a molten slag;
hot gas cleanup means to desulfurize said lean fuel gas;
cooling means to cool said syngas and said lean fuel gas;
activated carbon beds to remove Mercury from said syngas and from said lean fuel gas to produce clean syngas and clean lean fuel gas with said syngas being suitable to be used as a chemical or as a feedstock to produce liquid fuel such as methanol, gasoline, and dimethyl ether, and with said lean fuel gas being used to generate electric power in a combustion turbine with its hot exhaust containing N₂+CO₂ being fed to a boiler to raise steam and generate additional power;
means to direct the exhaust containing N₂+CO₂ from said boiler to said gasifier for the conversion of the N₂+CO₂ to N₂+2CO;
means to use the N₂+2CO as a feedstock; and
a fertilizer plant into which said feedstock is utilized to produce said fertilizer.

18. The apparatus as set forth in claim 16 wherein said pyrolyzation means possesses an intermediate manifold at its discharge adapted to divide said char into two parts, with the first part being fed to said gasifier to convert said char to lean fuel gas and the second part being fed to a quencher to cool said second part to such an extent that it cannot smoke prior to discharging it to the atmosphere.

19. The apparatus as set forth in claim 16 wherein said pyrolyzation means is adapted to combust a core of char within the pyrolyzation means to provide thermal energy to heat the coal within it.

20. The apparatus as set forth in claim 19 wherein said core is combusted with the aid of an injection lance adapted to inject a gas containing oxygen.

21. The apparatus as set forth in claim 20 wherein said gas containing oxygen is pure oxygen.

22. The apparatus as set forth in claim 19 wherein said core of char is surrounded by an annulus of coal.

23. The apparatus as set forth in claim 22 wherein said annulus of coal is adapted to being heated bi-directionally to efficiently devolatilize said coal and produce char.

24. The apparatus as set forth in claim 16 wherein said hot gas cleanup is adapted to produce elemental sulfur which is a valuable product.

25. The apparatus as set forth in claim 16 wherein said pyrolyzation means is adapted to operate under pressure to improve heat transfer.

26. The apparatus as set forth in claim 25 wherein said pyrolyzation means is equipped with a pushing ram at the charging end thereof to advance the contents within said pyrolyzation means to cause the charging of materials at the charging end and discharging char at the discharging end of said pyrolyzation means.

27. The apparatus as set forth in claim 25 wherein said pyrolyzation means is equipped with a mandrel with a bore at the charging end to produce a core through which an injection lance is free to independently advance and retract.
